(19) United States
(12) Patent Application Publication
(10) Pub. No.: US 2020/0345603 A1
(43) Pub. Date: Nov. 5, 2020

(54) PRODUCT FOR THE OXIDATIVE DYEING OF HAIR CONTAINING SPECIAL DEVELOPERS, COUPLERS AND ALKALIZING AGENTS

(71) Applicant: Henkel AG & Co. KGaA, Duesseldorf (DE)

(72) Inventors: KONSTANTIN GOUTSIS, Juechen (DE); DANIELA KESSLER-BECKER, Leverkusen (DE)

(73) Assignee: Henkel AG & Co. KGaA, Duesseldorf (DE)

(21) Appl. No.: 16/836,473

(22) Filed: Mar. 31, 2020

(30) Foreign Application Priority Data
Apr. 30, 2019 (DE) 10 2019 206 171.0

(51) Int. Cl. A61K 8/41; A61K 8/34; A61K 8/19; A61K 8/49; A61K 8/22; A61Q 5/10

(52) U.S. Cl. CPC A61K 8/41; A61K 8/411; A61K 8/347; A61K 2800/88; A61K 8/4926; A61K 8/22; A61Q 5/10; A61K 8/19

(57) ABSTRACT

Product for the oxidative dyeing of keratinous fibers, contained in a cosmetic carrier
(A) at least one developer selected from the group of p-toluenediamine, 2-(2-hydroxyethyl)-p-phenylenediamine, N,N-bis-(2-hydroxyethyl)-p-phenylenediamine, 2-methoxymethyl-p-phenylenediamine, p-aminophenol, 4-amino-3-methyl phenol, bis-(2-hydroxy-5-aminophenyl)-methane, 4,5-diamino-1-(2-hydroxyethyl)pyrazole and their physiologically compatible salts, and
(B) at least one coupler selected from the group of 1,5-dihydroxynaphthalene, 2,7-dihydroxynaphthalene, 1-naphthol, 1,7-dihydroxynaphthalene, 2,6-dihydroxy-3,4-dimethylpyridine, 2-amino-3-hydroxypyridine, 3-amino-2-methylamino-6-methoxypyridine, 3-aminophenol, 5-amino-2-methyl phenol, 3-amino-2-chloro-6-methyl phenol, 5-(2-hydroxyethyl)-amino-2-methyl phenol, 3-phenylenediamine, 2-(2,4-diaminophenoxy)ethanol, 1,3-bis(2,4-diaminophenoxy)propane, 1-methoxy-2-amino-4-(2-hydroxyethyl)amino)benzene, 2,6-bis(2′-hydroxyethylamino)-1-methylbenzene, resorcinol, 2-methylresorcinol, 4-chlororesorcinol and their physiologically compatible salts, and
(C) 2-dimethylamino-2-methyl-1-propanol.

### CROSS-REFERENCE TO RELATED APPLICATION

This application claims priority to German Patent Application No. 10 2019 206 171.0, filed Apr. 30, 2019, which is incorporated herein by reference in its entirety.

### TECHNICAL FIELD

The subject-matter of the present application is a product for the oxidative dyeing of keratinous fibers, in particular hair, containing the combination of a developer of a specific group (A), a coupler of a specific group (B) and 2-dimethylamino-2-methyl-1-propanol (C).

Further subject-matter of the application is a multi-component packaging unit (kit-of-parts or kit) comprising separately packaged the aforementioned product as well as an oxidizing agent preparation and a method for the oxidative dyeing of keratinous fibers, in which the aforementioned product or kit is used.

### BACKGROUND

Changing the color of keratinous fibers, especially of hair, is an important field of modern cosmetics. This allows the appearance of the hair to be adjusted to current fashion trends as well as to the individual wishes of the individual person. The expert knows various ways to change the color of hair. The hair color may be changed temporarily by using direct drawing dyes. In this process, already formed dyes diffuse from the dye into the hair fiber. Dyeing with direct drawing dyes is associated with a low level of hair damage, but a disadvantage is the low durability and the fast washability of the dyes obtained with direct drawing dyes.

If the consumer wants a long-lasting color result or a shade lighter than the original hair color, oxidative color modifiers are usually used. For permanent, intensive dyeing with corresponding fastness properties, so-called oxidative coloring agents are used. Such dyes usually contain oxidative dye precursors, so-called developer components and coupler components, which form the actual dyes when exposed to oxidizing agents. Oxidative dyes are exemplified by long-lasting dyeing results.

In order to achieve a satisfactory dyeing and brightening performance, oxidative dyeing or brightening agents generally require an alkaline pH value during application; especially at pH values from about 8.5 to about 10.5 optimum results are achieved.

The use of ammonia as an alkalizing agent for adjusting these pH values is known from the state of the art. Not only may ammonia be used to adjust the pH range necessary for the formation of dyes, but ammonia also causes considerable swelling of the hair. At the same time, ammonia acts as a penetrating agent or penetration aid. For these reasons, the use of ammonia in oxidative dyes generally results in more intensive dyeing compared to other alkalizing agents.

There are many application-related advantages associated with ammonia. However, these advantages are counterbalanced by a major disadvantage, because ammonia has a very unpleasant, pungent odor, which may be perceived as very unpleasant by the user.

Extensive efforts to reduce the odor of ammonia are already known from literature. For example, there is a large number of formulations that use a mixture of ammonia and monoethanolamine or exclusively monoethanolamine as alkalizing agent. However, it is also known from the state of the art that the complete or partial replacement of ammonia may often result in a poorer penetration of the dyes into the hair, which may result in poorer color intensity. For the development of particularly intensive shades, the use of monoethanolamine is therefore not an option.

It was therefore the task of the present disclosure to provide a product for oxidative dyeing of hair with reduced ammonia odor. If possible, the use of ammonia in the products should be avoided. Nevertheless, these products should not show any loss in their dyeing performance, i.e. ammonia-free dyes were sought which nevertheless allow the creation of particularly intensive color shades. Furthermore, the application of the product should not be associated with higher hair damage and the fastness properties of this product should also be particularly good.

### BRIEF SUMMARY

Products and methods for the oxidative dyeing of keratinous fibers are provided herein. In an embodiment, a product for the oxidative dyeing of keratinous fibers is provided, wherein the product is included in a cosmetic carrier. The product includes:

(A) at least one developer selected from the group of p-toluenediamine, 2-(2-hydroxyethyl)-p-phenylenediamine, N,N-bis-(2-hydroxyethyl)-p-phenylenediamine, 2-methoxymethyl-p-phenylenediamine, p-aminophenol, 4-amino-3-methyl phenol, bis-(2-hydroxy-5-aminophenyl)-methane, 4,5-diamino-1-(2-hydroxyethyl)pyrazole or their physiologically compatible salts, and

(B) at least one coupler selected from the group of 1,5-dihydroxynaphthalene, 2,7-dihydroxynaphthalene, 1-naphthol, 1,7-dihydroxynaphthalene, 2,6-dihydroxy-3,4-dimethylpyridine, 2-amino-3-hydroxypyridine, 3-amino-2-methylamino-6-methoxypyridine, 3-aminophenol, 5-amino-2-methyl phenol, 3-amino-2-chloro-6-methyl phenol, 5-(2-hydroxy-ethyl)-amino-2-methyl phenol, 3-phenylenediamine, 2-(2,4-diaminophenoxy)ethanol, 1,3-bis(2,4-diaminophenoxy)propane, 1-methoxy-2-amino-4-(2-hydroxyethyl)amino)benzene, 2,6-bis(2′-hydroxyethylamino)-1-methylbenzene, resorcinol, 2-methylresorcinol, 4-chlororesorcinol or their physiologically compatible salts, and

(C) 2-(dimethylamino)-2-methyl-1-propanol.

### DETAILED DESCRIPTION

The following detailed description is merely exemplary in nature and is not intended to limit the disclosure or the application and uses of the subject matter as described herein. Furthermore, there is no intention to be bound by any theory presented in the preceding background or the following detailed description.

Surprisingly, it has now turned out that this problem may be fully solved if a product is used in the dyeing of keratinous fibers which contains a developer selected from a specific group, a coupler selected from another specific group and, as alkalizing agent, 2-(dimethylamino)-2-methyl-1-propanol.

A first subject-matter of the present disclosure is a product for the oxidative dyeing of keratinous fibers, containing in a cosmetic carrier

(A) at least one developer selected from the group of p-toluenediamine, 2-(2-hydroxyethyl)-p-phenylenediamine, N,N-bis-(2-hydroxyethyl)-p-phenylenediamine, 2-methoxymethyl-p-phenylenediamine, p-aminophenol, 4-amino-3-methyl phenol, bis-(2-hydroxy-5-aminophenyl)-methane, 4,5-diamino-1-(2-hydroxyethyl)pyrazole and their physiologically compatible salts, and
(B) at least one coupler selected from the group of 1,5-dihydroxynaphthalene, 2,7-dihydroxynaphthalene, 1-naphthol, 1,7-dihydroxynaphthalene, 2,6-dihydroxy-3,4-dimethylpyridine, 2-amino-3-hydroxypyridine, 3-amino-2-methylamino-6-methoxypyridine, 3-aminophenol, 5-amino-2-methyl phenol, 3-amino-2-chloro-6-methyl phenol, 5-(2-hydroxy-ethyl)amino-2-methyl phenol, 3-phenylenediamine, 2-(2,4-diaminophenoxy)ethanol, 1,3-bis(2,4-diaminophenoxy)propane, 1-methoxy-2-amino-4-(2-hydroxyethyl)amino)benzene, 2,6-bis(2′-hydroxyethylamino)-1-methylbenzene, resorcinol, 2-methylresorcinol, 4-chlororesorcinol and their physiologically compatible salts, and
(C) 2-(dimethylamino)-2-methyl-1-propanol.

### Keratinous Fibers

Keratinous fibers, fibers containing keratin or keratin fibers are furs, wool, feathers and in particular human hair. Although the products as contemplated herein are primarily suitable for lightening and dyeing keratin fibers, there is nothing in principle to prevent their use in other areas.

### Product for Oxidative Dyeing of Keratinous Fibers

The term “product for oxidative dyeing” of keratin fibers used in the present disclosure refers to oxidative dyeing agents. Oxidative dyeing agents contain oxidative dye precursors, so-called developers and coupling components. Developers and couplers diffuse separately into the keratin fiber and form the actual dyes in chemical reaction with each other by employing an alkalizing agent and an oxidizing agent (usually hydrogen peroxide). Depending on the amount of the oxidizing agent used, the keratin fiber is simultaneously brightened to a greater or lesser extent during the dyeing process, since the oxidizing agent not only initiates the dye formation process of developers and couplers, but also oxidatively destroys the hair's own pigments (melanins).

Depending on the quantities of oxidative dye precursors and oxidizing agent used, oxidative dyeing may therefore be primarily dyeing (with a high dye content) or primarily brightening (with a high oxidizing agent content). In the latter case, the oxidative dye precursors are mainly used to nuance the brightening result. Both coloration and color shade are covered by the term “oxidative dyeing” as used in the present disclosure.

### Cosmetic Carriers

The products each contain the oxidative dye precursors essential to the present disclosure in a cosmetic carrier, preferably in a suitable aqueous, alcoholic or aqueous-alcoholic carrier. For the purpose of oxidative color change such carriers may be creams, emulsions, gels or also surfactant-containing foaming solutions such as shampoos, foaming aerosols, foam formulations or other preparations suitable for application on the hair. Especially preferred are products for the oxidative dyeing of keratinous fibers in creams or emulsions.

### Developers

As the first ingredient essential to the present disclosure, the products as contemplated herein contain at least one developer (A) of a specific group. The developer (A) may alternatively be designated as oxidative dye precursor of developer type (A).

These developer components form the actual dyes through the influence of oxidizing agents or atmospheric oxygen among themselves or through coupling with one or several coupler components.

The products as contemplated herein contain at least one developer (A) selected from the group of p-toluenediamine, 2-(2-hydroxyethyl)-p-phenylenediamine, N,N-bis-(2-hydroxyethyl)-p-phenylenediamine, 2-methoxymethyl-p-phenylenediamine, p-aminophenol, 4-amino-3-methyl phenol, bis-(2-hydroxy-5-aminophenyl)methane, 4,5-diamino-1-(2-hydroxyethyl)pyrazole and their physiologically compatible salts.

The developers may be used in the product either as free compounds or in the form of one of their physiologically compatible salts.

A physiologically compatible salt is defined as a salt which is well tolerated by the body under physiological conditions, i.e. a physiologically compatible salt may be safely applied on the head of a user and brought into contact with the scalp during the process of oxidative hair dyeing. If the developer is used in the form of its physiologically compatible salt, at least one of its amino groups is protonated and neutralized by one or several anions while maintaining electron neutrality, which are preferably selected from the group of chloride, sulfate, hydrogen sulfate, bromide, benzene sulfonate, p-toluenesulfonate, C₁-C₄ alkane sulfonate, C₁-C₄ alkane sulfate, trifluoromethane sulfonate and acetate, lactate, citrate and/or tartrate.

P-toluenediamine is a compound of the formula

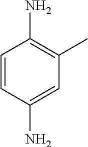

Preferred physiologically compatible salts of p-toluenediamine are in particular the hydrochlorides (monohydrochloride×HCl, or dihydrochloride×2 HCl), the sulfate (×H₂SO₄) and the hydrobromides (monohydrobromide×HBr, or dihydrobromide×2 HBr) of the compound. Especially preferred is p-toluenediamine sulfate.

2-(2-hydroxyethyl)-p-phenylenediamine may alternatively also be designated as 2-(2,5-diaminophenyl)ethanol. This is the compound of formula

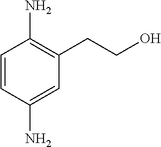 [SMILES: Nc1ccc(N)c(CCO)c1]

Preferred physiologically compatible salts of 2-(2,5-diaminophenyl)ethanol are in particular the hydrochlorides (monohydrochloride×HCl, or dihydrochloride×2 HCl), the sulfate (×H₂SO₄) and the hydrobromides (monohydrobromide×HBr, or dihydrobromide×2 HBr) of the compound. Particularly preferred is 2-(2,5-diaminophenyl)ethanol sulfate

N,N-bis-(2-hydroxyethyl)-p-phenylenediamine is a compound of the formula

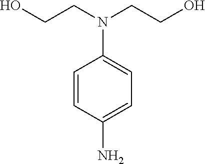 [SMILES: Nc1ccc(N(CCO)CCO)cc1]

Preferred physiologically compatible salts of N,N-bis-(2-hydroxyethyl)-p-phenylenediamine are in particular the hydrochlorides (monohydrochloride×HCl, or dihydrochloride×2 HCl), the sulfate (×H₂SO₄) and the hydrobromides (monohydrobromide×HBr, or dihydrobromide×2 HBr) of the compound. N,N-bis-(2-hydroxyethyl)-p-phenylenediamine sulfate is particularly preferred.

2-methoxymethyl-p-phenylenediamine is a compound of the formula

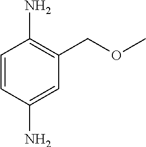

Preferred physiologically compatible salts of 2-methoxymethyl-p-phenylenediamine are in particular the hydrochlorides (monohydrochloride×HCl, or dihydrochloride×2 HCl), the sulfate (×H₂SO₄) and the hydrobromides (monohydrobromide×HBr, or dihydrobromide×2 HBr) of the compound 2-methoxymethyl-p-phenylenediamine sulfate is particularly preferred.

P-aminophenol is a compound of the formula

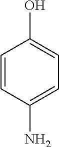

Preferred physiologically compatible salts of p-aminophenol are in particular the hydrochloride (monohydrochloride×HC), the sulfate (×½ H₂SO₄) and the hydrobromide (monohydrobromide×HBr) of the compound. Especially preferred is p-aminophenol sulfate (×½ H₂SO₄).

4-amino-3-methyl phenol is a compound of the formula

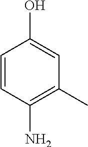

Preferred physiologically compatible salts of 4-amino-3-methyl phenol are in particular the hydrochloride (monohydrochloride×HC), the sulfate (×½ H₂SO₄) and the hydrobromide (monohydrobromide×HBr) of the compound. Especially preferred is 4-amino-3-methyl phenol sulfate (×½ H₂SO₄).

Bis-(2-hydroxy-5-aminophenyl)methane is a compound of the formula

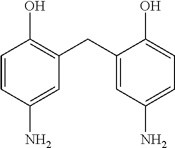 [SMILES: Nc1ccc(O)c(Cc2cc(N)ccc2O)c1]

Preferred physiologically compatible salts of bis-(2-hydroxy-5-aminophenyl)methane are especially the hydrochlorides (monohydrochloride×HCl, dihydrochloride×2 HCl, trihydrochloride×3 HCl or the tetrahydrochloride×4 HCl), the sulfates (×H₂SO₄ or ×2H₂SO₄) and the hydrobromides (monohydrobromide×HBr, or dihydrobromide×2 HBr, trihydrochloride×3 HCl or tetrahydrobromide×4 HBr) of the compound. Bis-(2-hydroxy-5-aminophenyl)methane tetrahydrochloride is particularly preferred.

4,5-diamino-1-(2-hydroxyethyl)-1H-pyrazole is the compound of the formula

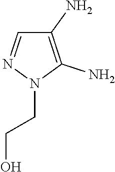

Preferred physiologically compatible salts of 4,5-diamino-1-(2-hydroxyethyl)-1H-pyrazole are in particular the hydrochlorides (monohydrochloride×HCl, or dihydrochloride×2 HCl), the sulfate (×H₂SO₄), and the hydrobromides (monohydrobromide×HBr, or dihydrobromide×2 HBr) of the compound. Particularly preferred is 4,5-diamino-1-(2-hydroxyethyl)-1H-pyrazole sulfate.

In the course of the work leading to this present disclosure, it was discovered that the use of very specific developers from group (A) enables the dyeing of keratin fibers in very intensive colors.

Colorations with the highest color intensity could be obtained when the keratin fibers were dyed with a product containing as developer (A) p-toluenediamine, 2-(2-hydroxyethyl)-p-phenylenediamine and/or one of their physiologically compatible salts.

Within the scope of a particularly preferred embodiment, a product as contemplated herein includes

(A) at least one developer selected from the group of p-toluenediamine, 2-(2-hydroxyethyl)-p-phenylenediamine and their physiologically compatible salts.

Good color intensities could also be produced on keratinous fibers if the product as contemplated herein contained as developer (A) a developer from the group of p-aminophenol, 4-amino-3-methyl phenol and their physiologically compatible salts.

Within the scope of a further preferred embodiment, a product as contemplated herein includes

(A) at least one developer from the group of p-aminophenol, 4-amino-3-methyl phenol and their physiologically compatible salts.

The quantities of these developers from group (A) in the product as contemplated herein will be selected by the expert depending on the desired color intensity and color depth. Good color results are already obtained from an application concentration of about 0.01% by weight. If particularly intensive color nuances are to be developed, the expert will select correspondingly high application concentrations of, for example, about 3.5% by weight or up to about 5.0% by weight. All figures in % by weight refer to the total amount of developer (A) contained in the product in relation to the total weight of the product.

In the context of a further preferred embodiment, a product as contemplated herein includes—based on the total weight of the product—one or several developers (A) in a total amount of from about 0.01 to about 5.0% by weight, preferably of from about 0.1 to about 3.5% by weight, more preferably of from about 0.3 to about 1.5% by weight and very particularly preferably of from about 0.5 to about 0.8% by weight.

For the purpose of further nuance, the products as contemplated herein may contain, in addition to the developers (A) which are essential to the present disclosure, one or several further developers which are different from the developers of group (A). These may, for example, be selected from the group formed by 2-(1,2-dihydroxyethyl)-p-phenylenediamine, N-(4-amino-3-methylphenyl)-N-[3-(1H-imidazol-1-yl)propyl]amine, N,N′-bis-(2-hydroxyethyl)-N,N′-bis-(4-aminophenyl)-1,3-diaminopropan-2-ol, 1,3-bis-(2,5-diaminophenoxy)propan-2-ol, N,N′-bis-(4-aminophenyl)-1,4-diazacycloheptane, 1,10-bis-(2,5-diaminophenyl)-1,4,7,10-tetraoxadecane, 4-amino-2-aminomethyl phenol, 4-amino-2-(1,2-dihydroxyethyl)phenol, 4-amino-2-(diethylaminomethyl)phenol, 2,4,5,6-tetraaminopyrimidine, 4-hydroxy-2,5,6-triaminopyrimidine, 2-hydroxy-4,5,6-triaminopyrimidine, 2,3-diamino-6,7-dihydro-1H,5H-pyrazolo[1,2-a]pyrazol-1-on and their physiologically compatible salts.

These further developers, which are different from the developers from group (A), may be contained in the product as contemplated herein in a total amount from about 0.01 to about 4.0% by weight, preferably from about 0.1 to about 3.5% by weight, further preferably from about 0.6 to about 3.1% by weight and very particularly preferably from about 1.2 to about 2.2% by weight, based on the total weight of the product as contemplated herein.

### Coupler

As a second ingredient essential to the present disclosure, the products as contemplated herein contain at least one coupler (B) of a specific group. The coupler (B) may alternatively also be designated as oxidative dye precursors of coupler type (B).

Coupler components alone do not form a significant coloration in the context of oxidative dyeing, but always require the presence of developer components. Coupler components as contemplated herein allow at least one substitution of a chemical residue of the coupler by the oxidized form of the developer component. Covalent bonds are formed between the coupler and developer components.

The products as contemplated herein contain at least one coupler (B) selected from the group of 1,5-dihydroxynaphthalene, 2,7-dihydroxynaphthalene, 1-naphthol, 1,7-dihydroxynaphthalene, 2,6-dihydroxy-3,4-dimethylpyridine, 2-amino-3-hydroxypyridine, 3-amino-2-methylamino-6-methoxypyridine, 3-aminophenol, 5-amino-2-methyl phenol, 3-amino-2-chloro-6-methyl phenol, 5-(2-hydroxyethyl)-amino-2-methyl phenol, 3-phenylenediamine, 2-(2,4-diaminophenoxy)ethanol, 1,3-bis(2,4-diaminophenoxy)propane, 1-methoxy-2-amino-4-(2-hydroxyethylamino)benzene, 2,6-bis(2′-hydroxyethyl amino)-1-methylbenzene, resorcinol, 2-methylresorcinol, 4-chlororesorcinol and their physiologically compatible salts

The couplers are preferably used in the form of their free compound. If the coupler contains at least one amino group, however, it is also possible to use the coupler in the form of a physiologically compatible salt in the product as contemplated herein.

If the coupler is used in the form of its physiologically compatible salt, at least one of its amino groups is present in protonated form and is neutralized by one or several anions while maintaining electron neutrality, which are preferably selected from the group of chloride, sulfate, hydrogen sulfate, bromide, benzene sulfonate, p-toluenesulfonate, C₁-C₄ alkane sulfonate, C₁-C₄ alkane sulfate, trifluoromethane sulfonate and acetate, lactate, citrate and/or tartrate.

1,5-dihydroxynaphthalene may alternatively also be referred to as naphthalene-1,5-diol and has the CAS number 83-56-7.

2,7-dihydroxynaphthalene may alternatively also be referred to as naphthalene-2,7-diol and has the CAS number 582-17-2.

1-naphthol has the CAS number 90-15-3.

1,7-dihydroxynaphthalene may alternatively be called naphthalene-1,7-diol and has the CAS number 575-38-2.

2,6-dihydroxy-3,4-dimethylpyridine is alternatively referred to as Ro598 or Colipa A99 and has the CAS number 84540-47-6.

2-amino-3-hydroxypyridine is alternatively also referred to as COLIPA no. A132 and has the CAS number 16867-03-1.

3-amino-2-methylamino-6-methoxypyridine is alternatively also referred to as COLIPA no. A130 and has the CAS numbers 90817-34-8 (in the form of the hydrochloride) and 83732-72-3 (in the form of the dihydrochloride).

3-aminophenol is also alternatively referred to as m-aminophenol and has the CAS numbers 591-27-5 (as free compound), 51-81-0 (as hydrochloride) and 68239-81-6 (as sulfate×½ H₂SO₄).

5-amino-2-methyl phenol is alternatively also referred to as 4-amino-2-hydroxytoluene or as 4-amino-2-hydroxytoluene or as COLIPA no. A27 and has the CAS number 2835-95-2.

3-Amino-2-chloro-6-methyl phenol is alternatively also referred to as 5-amino-6-chloro-o-cresol or as 6-methyl-3-amino-2-chlorophenol and has the CAS numbers 84540-50-1 (free base) and 80419-48-3 (hydrochloride).

5-(2-hydroxyethyl)-amino-2-methyl phenol is alternatively referred to as 2-hydroxy-4-(b-hydroxyethyl)-aminotoluene or as Colipa no. A31 and has the CAS number 55302-96-0.

3-phenylenediamine is alternatively also referred to as m-phenylenediamine or 1,3-diaminobenzene and has the CAS number 108-45-2.

2-(2,4-diaminophenoxy)ethanol is alternatively also referred to as IMEXINE OAJ (dihydrochloride) or COLIPA A42 and has the CAS numbers 70643-19-5 (free base), 66422-95-5 (dihydrochloride) and 70643-20-8 (sulfate).

1,3-bis(2,4-diaminophenoxy)propane is alternatively also referred to as 4,4′-[1,3-propanediylbis(oxy)]bis-1,3-benzenediamine and has CAS numbers 81892-72-0 (free base) and 74918-21-1 (tetrahydrochloride).

1-methoxy-2-amino-4-(2-hydroxyethylamino)benzene is alternatively also referred to as 2-[(3-amino-4-methoxyphenyl)amino]ethanol or as Colipa no. A84 and has the CAS numbers 83763-47-7 (free base) and 83763-48-8 (sulfate).

2,6-bis(2′-hydroxyethylamino)-1-methylbenzene is also alternatively referred to as 2,6-bis[(2-hydroxyethyl)-amino]toluene or as COLIPA no. A138 and has the CAS number 149330-25-6.

Resorcinol is alternatively also referred to as 1,3-dihydroxybenzene or m-dihydroxybenzene and has the CAS number 108-46-3.

2-methylresorcinol is alternatively referred to as 2-methyl-1,3-benzenediol or 2,6-dihydroxytoluenes and has the CAS number 608-25-3.

4-Chlororesorcinol is alternatively also referred to as 4-chloro-1,3-benzenediol and has the CAS number 95-88-5.

In the course of the work leading to this present disclosure, it has been found that the use of certain couplers from group (B) leads to particularly intensive color results. The best results could be obtained when the products as contemplated herein contained as couplers (B) at least one naphthol derivative with at least one hydroxy group. Corresponding naphthol derivatives are 1,5-dihydroxynaphthalene, 2,7-dihydroxynaphthalene, 1-naphthol and 1,7-dihydroxynaphthalene.

Within the context of an explicitly particularly preferred embodiment, a product as contemplated herein is exemplified by the fact that it includes

(B) at least one coupler selected from the group of 1,5-dihydroxynaphthalene, 2,7-dihydroxynaphthalene, 1-naphthol and 1,7-dihydroxynaphthalene.

The very best results were obtained with the 1,5-dihydroxynaphthalene coupler. This coupler from group (B) is the most preferred.

Within the context of an explicitly particularly preferred embodiment, a product as contemplated herein is exemplified by the fact that it includes

(B) 1,5-dihydroxynaphthalene as coupler.

Even with heterocyclic couplers (B), intensively dyed keratin fibers could be obtained if they were applied to the keratin fiber in the dyeing process in combination with the special developers of group (A) and 2-(dimethylamino)-2-methyl-1-propanol (C). Corresponding heterocyclic couplers are 2,6-dihydroxy-3,4-dimethylpyridine, 2-amino-3-hydroxypyridine, 3-amino-2-methylamino-6-methoxypyridine or their physiologically compatible salts.

Within the context of a further explicitly particularly preferred embodiment, a product as contemplated herein is exemplified by the fact that it includes

(B) at least one coupler selected from the group of 2,6-dihydroxy-3,4-dimethylpyridine, 2-amino-3-hydroxypyridine, 3-amino-2-methylamino-6-methoxypyridine and physiologically compatible salts thereof.

Within the group of heterocyclic couplers (B), 2,6-dihydroxy-3,4-dimethylpyridine is the most preferred.

Within the context of a further explicitly particularly preferred embodiment, a product as contemplated herein is exemplified by the fact that it includes

(B) 2,6-dihydroxy-3,4-dimethylpyridine as coupler.

Good results could also be obtained with couplers (B) from the group of m-aminophenols when used in the dyeing process in combination with the special developers of group (A) and 2-(dimethylamino)-2-methyl-1-propanol (C) on the keratin fiber. Corresponding aminophenols are 3-aminophenol, 5-amino-2-methyl phenol, 3-amino-2-chloro-6-methyl phenol, 5-(2-hydroxyethyl)-amino-2-methyl phenol or the physiologically compatible salts thereof.

Within the context of a further explicitly particularly preferred embodiment, a product as contemplated herein is exemplified by the fact that it includes

(B) at least one coupler from the group of 3-aminophenol, 5-amino-2-methyl phenol, 3-amino-2-chloro-6-methyl phenol, 5-(2-hydroxyethyl)-amino-2-methyl phenol and their physiologically compatible salts.

Good results could also be obtained with couplers (B) from the group of m-phenylenediamines when used in the dyeing process in combination with the special developers of group (A) and 2-(dimethylamino)-2-methyl-1-propanol (C) on the keratin fiber. The corresponding m-phenylenediamines are 3-phenylenediamine, 2-(2,4-diaminophenoxy)ethanol, 1,3-bis(2,4-diaminophenoxy)propane, 1-methoxy-2-amino-4-(2-hydroxyethylamino)benzene, 2,6-bis(2′-hydroxyethyl amino)-1-methylbenzene or the physiologically compatible salts thereof.

Within the context of a further explicitly particularly preferred embodiment, a product as contemplated herein is exemplified by the fact that it includes

(B) at least one coupler selected from the group of 3-phenylenediamine, 2-(2,4-diaminophenoxy)ethanol, 1,3-bis(2,4-diaminophenoxy)propane, 1-methoxy-2-amino-4-(2-hydroxyethylamino)benzene, 2,6-bis(2′-hydroxyethylamino)-1-methylbenzene, and physiologically compatible salts thereof.

Good results were also obtained with couplers (B) from the group of m-dihydroxybenzenes when used in the dyeing process in combination with the special developers (A) and 2-dimethylamino-2-methyl-1-propanol (C) on the keratin fiber. Corresponding m-dihydroxybenzenes are resorcinol, 2-methylresorcinol, 4-chlororesorcinol.

Within the context of a further explicitly particularly preferred embodiment, a product as contemplated herein is exemplified by the fact that it includes

(B) at least one coupler selected from the group of resorcinol, 2-methylresorcinol, 4-chlororesorcinol.

The quantities of couplers from group (B) used in the product as contemplated herein will be selected by the expert depending on the desired color intensity and color depth. Good color results are already obtained from an application concentration of about 0.01% by weight. If particularly intensive color nuances are to be developed, the expert will select correspondingly high application concentrations of, for example, about 3.5% by weight or up to about 5.0% by weight. All data in % by weight refer to the total amount of coupler (B) contained in the product, which is put in relation to the total weight of the agent.

Within the context of a further explicitly particularly preferred embodiment, a product as contemplated herein is exemplified by the fact that it includes—based on the total weight of the composition—one or several couplers (B) in a total amount from about 0.01 to about 5.0% by weight, preferably from about 0.1 to about 3.5% by weight, further preferably from about 0.3 to about 1.5% by weight and very particularly preferably from about 0.4 to about 0.8% by weight.

For further color shading, in addition to couplers (B) essential to the present disclosure, the products as contemplated herein may also contain one or several further couplers which are different from the couplers of group (B). These may, for example, be selected from the group formed by 2-hydroxy-4-aminophenoxyethanol, 5-amino-4-chloro-2-methyl phenol, 5-(2-hydroxyethyl)-amino-2-methyl phenol, 2,4-dichloro-3-aminophenol, 2-aminophenol, 2-({3-[(2-hydroxyethyl)amino]-4-methoxy-5-methylphenyl}amino)ethanol, 2-({3-[(2-hydroxyethyl)amino]-2-methoxy-5-methylphenyl}amino)ethanol, 2-({3-[(2-hydroxyethyl)amino]-4,5-dimethylphenyl}amino)ethanol, 2-[3-morpholin-4-ylphenyl)amino]ethanol, 3-amino-4-(2-methoxyethoxy)-5-methylphenylamine, 1-amino-3-bis-(2-hydroxyethyl)amino benzene, 1,2,4-trihydroxybenzene, 3,5-diamino-2,6-dimethoxypyridine, 1-phenyl-3-methylpyrazol-5-on, 1,8-dihydroxynaphthalene, 4-hydroxy-indole, 6-hydroxyindole, 7-hydroxyindole, 4-hydroxyindoline, 6-hydroxyindoline, 7-hydroxyindoline or mixtures of these compounds or their physiologically compatible salts.

These further couplers, which are different from the couplers from group (B), may be present in the product as contemplated herein in a total amount from about 0.01 to about 4.0% by weight, preferably from about 0.1 to about 3.5% by weight, further preferably from about 0.6 to about 3.1% by weight and very particularly preferably from about 1.2 to about 2.2% by weight, based on the total weight of the product as contemplated herein.

### Direct Drawing Dyes

In addition to the developers (A) and the couplers (B), the products as contemplated herein may also contain at least one direct drawing dye from the group of anionic, non-ionic and/or cationic dyes.

Especially preferred are one or several non-ionic direct drawing dyes from the group of HC Yellow 2, HC Yellow 4, HC Yellow 5, HC Yellow 6, HC Yellow 12, HC Orange 1, Disperse Orange 3, HC Red 1, HC Red 3, HC Red 7, HC Red 10, HC Red 11, HC Red 13, HC Red BN, HC Blue 2, HC Blue 11, HC Blue 12, Disperse Blue 3, HC Violet 1, Disperse Violet 1, Disperse Violet 4, Disperse Black 9,1,4-diamino-2-nitrobenzene, 2-amino-4-nitrophenol, 1,4-bis(2-hydroxyethyl)-amino-2-nitrobenzene, 3-nitro-4-(2-hydroxyethyl)aminophenol, 2-(2-hydroxyethyl)amino-4,6-dinitrophenol, 4-[(2-hydroxyethyl)amino]-3-nitro-1-methylbenzene, 1-amino-4-(2-hydroxyethyl)amino-5-chloro-2-nitrobenzene, 4-amino-3-nitrophenol, 1-(2′-ureidoethyl)amino-4-nitrobenzene, 2-[(4-amino-2-nitrophenyl)amino]benzoic acid, 4-[(3-hydroxypropyl)amino]-3-nitrophenol, 4-nitro-o-phenylenediamine, 6-nitro-1,2,3,4-tetrahyldroquinoxaline, 2-hydroxy-1,4-naphthoquinone, picramic acid and its salts, 2-amino-6-chloro-4-nitrophenol, 4-ethylamino-3-nitrobenzoic acid and 2-chloro-6-ethylamino-4-nitrophenol.

Within the context of a further explicitly particularly preferred embodiment, a product as contemplated herein is exemplified by the fact that it includes additionally one or several non-ionic direct drawing dyes from the group of HC Yellow 2, HC Yellow 4, HC Yellow 5, HC Yellow 6, HC Yellow 12, HC Orange 1, Disperse Orange 3, HC Red 1, HC Red 3, HC Red 7, HC Red 10, HC Red 11, HC Red 13, HC Red BN, HC Blue 2, HC Blue 11, HC Blue 12, Disperse Blue 3, HC Violet 1, Disperse Violet 1, Disperse Violet 4, Disperse Black 9,1,4-diamino-2-nitrobenzene, 2-amino-4-nitrophenol, 1,4-bis(2-hydroxyethyl)-amino-2-nitrobenzene, 3-nitro-4-(2-hydroxyethyl)aminophenol, 2-(2-hydroxyethyl)amino-4,6-dinitrophenol, 4-[(2-hydroxyethyl)amino]-3-nitro-1-methylbenzene, 1-amino-4-(2-hydroxy-ethyl)amino-5-chlor-2-nitrobenzol, 4-amino-3-nitrophenol, 1-(2′-ureidoethyl)amino-4-nitrobenzene, 2-[(4-amino-2-nitrophenyl)amino]benzoic acid, 4-[(3-hydroxypropyl)amino]-3-nitrophenol, 4-nitro-o-phenylenediamine, 6-nitro-1,2,3,4-tetrahyldroquinoxaline, 2-hydroxy-1,4-naphthoquinone, picramic acid and its salts, 2-amino-6-chloro-4-nitrophenol, 4-ethylamino-3-nitrobenzoic acid and 2-chloro-6-ethylamino-4-nitrophenol.

In addition, anionic direct drawing dyes may also be included which are known under the international designations or trade names Acid Yellow 1, Yellow 10, Acid Yellow 23, Acid Yellow 36, Acid Orange 7, Acid Red 33, Acid Red 52, Pigment Red 57:1, Acid Blue 7, Acid Green 50, Acid Violet 43, Acid Black 1, Acid Black 52, bromophenol blue and tetrabromophenol Blue.

Suitable cationic direct drawing dyes are cationic triphenylmethane dyes, such as Basic Blue 7, Basic Blue 26, Basic Violet 2 and Basic Violet 14, aromatic systems substituted with a quaternary nitrogen group, such as Basic Yellow 57, Basic Red 76, Basic Blue 99, Basic Brown 16 and Basic Brown 17, cationic anthraquinone dyes such as HC Blue 16 (Bluequat B) and direct drawing dyes containing a heterocycle containing at least one quaternary nitrogen atom, in particular Basic Yellow 87, Basic Orange 31 and Basic Red 51. The cationic direct drawing dyes marketed under the trademark Arianor are also suitable cationic direct drawing dyes as contemplated herein.

The optionally additionally contained direct drawing dyes may, for example, be used in an amount of from about 0.0001 to about 5.0% by weight, preferably from about 0.001 to about 3.5% by weight, each based on the total weight of the present disclosure.

### 2-(dimethylamino)-2-methyl-1-propanol

As the third essential component (C) of the present disclosure, the products as contemplated herein contain 2-(dimethylamino)-2-methyl-1-propanol.

2-(dimethylamino)-2-methyl-1-propanol is an alkanolamine of the formula (C-1)

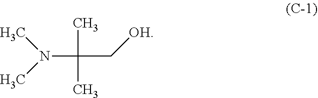

2-(dimethylamino)-2-methyl-1-propanol has the CAS number 7005-47-2 and may be purchased commercially from regular chemical suppliers such as Sigma-Aldrich, TCI Deutschland GmbH or ABCR GmbH Product List.

The work leading to this present disclosure has shown that the product as contemplated herein may be adjusted easily, quickly and precisely to the alkaline pH value required for the oxidative dyeing process with 2-(dimethylamino)-2-methyl-1-propanol. Furthermore, it has been shown in this context that this special alkalizing agent in combination with the aforementioned developers (A) and couplers (B) leads to particularly intensive dyeing on the keratin fibers. Surprisingly, the colorations obtained in this combination with 2-(dimethylamino)-2-methyl-1-propanol were even superior to the colorations obtained when ammonia was used as alkalizing agent.

For the creation of particularly intensive dyeing results, it has proved to be particularly advantageous to use dimethylamino-2-methyl-1-propanol (C) in certain quantity ranges in the product as contemplated herein. It is particularly preferred if the product contains—based on the total weight of the product—from about 0.5 to about 25.0% by weight, preferably from about 2.0 to about 20.0% by weight, more preferably from about 4.0 to about 18.0% by weight and very preferably from about 8.0 to about 14.0% by weight of 2-dimethylamino-2-methyl-1-propanol (C).

In the context of a further preferred embodiment, a product as contemplated herein includes—based on the total weight of the product—from about 0.5 to about 25.0% by weight, preferably from about 2.0 to about 20.0% by weight, further preferably from about 4.0 to about 18.0% by weight and very particularly preferably from about 8.0 to about 14.0% by weight of 2-dimethylamino-2-methyl-1-propanol (C).

### Further Alkalizing Agents

In principle, in addition to 2-dimethylamino-2-methyl-1-propanol (C), at least one further alkalizing agent may be used in the product as contemplated herein. Suitable further alkalizing agents may typically be selected from the group of inorganic salts, organic alkalizing agents other than 2-dimethylamino-2-methyl-1-propanol (C), basic amino acids and ammonia.

Organic alkalizing agents usable as contemplated herein may be selected, for example, from the group of alkanolamines of primary, secondary or tertiary amines with a C₂-C₆ alkyl base which carries at least one hydroxyl group, such as 2-aminoethan-1-ol (monoethanolamine), 2-amino-2-methyl-1-propanol, 2-amino-2-methyl-1-propanol, 2-amino-2-methyl-1,3-diol and triethanolamine

Inorganic alkalizing agents which may be used as contemplated herein may, for example, be selected from the group formed by sodium hydroxide, potassium hydroxide, calcium hydroxide, barium hydroxide, sodium phosphate, potassium phosphate, sodium silicate, potassium silicate, sodium carbonate and potassium carbonate.

The basic amino acids usable as alkalizing agents as contemplated herein may be selected from the group formed by L-arginine, D-arginine, D/L-arginine, L-lysine, D-lysine, D/L-lysine, L-histidine, D-histidine and D/L-histidine.

However, since the use of 2-dimethylamino-2-methyl-1-propanol (C) in the product as contemplated herein enables the creation of particularly intensive color results, it is of particular advantage to dispense with further alkalizing agents, especially ammonia, as in this way the odor nuisance caused by the ammonia may be minimized or even completely avoided.

The products as contemplated herein therefore contain—based on their total weight—less than about 1.75% by weight, preferably less than about 1.0% by weight, more preferably than about 0.5% by weight and very particularly preferably less than about 0.1% by weight of ammonia. The basis for these calculations is ammonia calculated as NH₃ (molar mass=17.03 g/mol).

Within the context of a further explicitly particularly preferred embodiment, a product as contemplated herein is exemplified by the fact that it includes—based on the total weight of the product—less than about 1.75% by weight, preferably less than about 1.0% by weight, more preferably than about 0.5% by weight and very particularly preferably less than about 0.1% by weight of ammonia.

### pH Value

2-dimethylamino-2-methyl-1-propanol (C) is used to adjust the alkaline pH value in the product as contemplated herein which is necessary for the oxidative dyeing process, whereby the preferred and particularly preferred quantity ranges may be selected.

For fine adjustment of the alkaline pH value, the product as contemplated herein may also contain one or several acidifying agents in small quantities. As contemplated herein, the preferred acidifying agents are food-grade acids, such as citric acid, acetic acid, malic acid or tartaric acid, as well as diluted mineral acids. The pH values in the sense of the present disclosure are pH values measured at a temperature of 22° C.

It has been found to be particularly preferred if the product as contemplated herein is based on an aqueous or aqueous cosmetic carrier and is adjusted to a pH value of from about 8.0 to about 12.0, preferably of from about 8.5 to about 11.5, further preferably of from about 9.0 to about 11.0 and particularly preferably of from about 9.5 to about 10.5.

Within the context of a further explicitly particularly preferred embodiment, a product as contemplated herein is exemplified by the fact that it includes water and has a pH value of from about 8.0 to about 12.0, preferably of from about 8.5 to about 11.5, further preferably of from about 9.0 to about 11.0 and very particularly preferably of from about 9.5 to about 10.5.

### Further Formulation Components

The product as contemplated herein may optionally contain other active ingredients, auxiliary substances and additives. In particular, the product as contemplated herein also contains at least one further ingredient from the group of fat components, surfactants and/or solvents.

Within the context of a further explicitly particularly preferred embodiment, a product as contemplated herein is exemplified by the fact that it additionally includes at least one further ingredient from the group of fat constituents, surfactants and/or solvents.

For the purposes of the present disclosure, “fat constituents” are organic compounds with a solubility in water at room temperature (22° C.) and atmospheric pressure (about 760 mmHg) of less than about 1% by weight, preferably less than about 0.1% by weight.

The definition of fat constituents explicitly covers only uncharged (i.e. non-ionic) compounds. Fat constituents have at least one saturated or unsaturated alkyl group with at least 8 C atoms. The molar weight of the fat components is a maximum of about 5000 g/mol, preferably a maximum of about 2500 g/mol and particularly preferably a maximum of about 1000 g/mol. The fat components are neither polyoxyalkylated nor polyglycerylated compounds.

Preferred fat constituents may be understood from the group of C₁₂-C₃₀ fatty alcohols, C₁₂-C₃₀ fatty acid triglycerides, C₁₂-C₃₀ fatty acid monoglycerides, C₁₂-C₃₀ fatty acid diglycerides and/or hydrocarbons. For the purposes of the present disclosure, only non-ionic substances are explicitly considered as fat components. Charged compounds such as fatty acids and their salts are not understood as fat constituents.

C₁₂-C₃₀ fatty alcohols may be saturated, mono- or polyunsaturated, linear or branched fatty alcohols with from about 12 to about 30 C atoms.

Examples of preferred linear, saturated C₁₂-C₃₀ fatty alcohols are dodecan-1-ol (dodecyl alcohol, lauryl alcohol), tetradecan-1-ol (tetradecyl alcohol, myristyl alcohol), hexadecan-1-ol (hexadecyl alcohol, cetyl alcohol, palmityl alcohol), octadecan-1-ol (octadecyl alcohol, stearyl alcohol), arachyl alcohol (eicosan-1-ol), heneicosyl alcohol (heneicosan-1-ol) and/or behenyl alcohol (docosan-1-ol).

Preferred linear unsaturated fatty alcohols are (9Z)-octadec-9-en-1-ol (oleyl alcohol), (9E)-octadec-9-en-1-ol (elaidyl alcohol), (9Z,12Z)-octadeca-9,12-dien-1-ol (linoleyl alcohol), (9Z,12Z,15Z)-octadeca-9,12,15-trien-1-ol (linolenoyl alcohol), gadoleyl alcohol ((9Z)-eicos-9-en-1-ol), arachidone alcohol ((5Z,8Z,11Z,14Z)-eicosa-5,8,11,14-tetraen-1-ol), erucyl alcohol ((13Z)-docos-13-en-1-ol) and/or brassidyl alcohol ((13E)-docosen-1-ol).

The preferred representatives for branched fatty alcohols are 2-octyl-dodecanol, 2-hexyl-dodecanol and/or 2-butyl-dodecanol.

For the purposes of the present disclosure, a C₁₂-C₃₀ fatty acid triglyceride is understood to be the triester of trivalent alcohol glycerol with three equivalents of fatty acid. Both fatty acids of the same structure and different fatty acids within a triglyceride molecule may be included in the formation of esters.

As contemplated herein, fatty acids are saturated or unsaturated, unbranched or branched, unsubstituted or substituted C₁₂-C₃₀ carboxylic acids. Unsaturated fatty acids may be mono- or polyunsaturated. In the case of an unsaturated fatty acid, its C—C double bond(s) may have the cis or trans configuration.

The fatty acid triglycerides are particularly suitable for this purpose, in which at least one of the ester groups is formed from glycerol with a fatty acid selected from dodecanoic acid (lauric acid), tetradecanoic acid (myristic acid), hexadecanoic acid (palmitic acid), tetracosanoic acid (lignoceric acid), octadecanoic acid (stearic acid), eicosanoic acid (arachidic acid), docosanoic acid (behenic acid), petroselinic acid [(Z)-6-octadecenoic acid], palmitoleic acid [(9Z)-hexadec-9-enoic acid], oleic acid [(9Z)-octadec-9-enoic acid], elaidic acid [(9E)-octadec-9-enoic acid], erucic acid [(13Z)-docos-13-enoic acid], linoleic acid [(9Z,12Z)-octadeca-9,12-diene acid, linolenic acid [(9Z,12Z,15Z)-octadeca-9,12,15-trienoic acid, elaeostearic acid [(9Z,11E,13E)-octadeca-9,11,3-trienoic acid], arachidonic acid [(5Z,8Z,11Z,14Z)-cosa-5,8,11,14-tetraenoic acid] and/or nervonic acid [(15Z)-tetracos-15-enoic acid].

The fatty acid triglycerides may also be of natural origin. The fatty acid triglycerides or mixtures thereof occurring in soybean oil, peanut oil, olive oil, sunflower oil, macadamia nut oil, moringa oil, apricot kernel oil, marula oil and/or possibly hardened castor oil are particularly suitable for use in the product as contemplated herein.

A C₁₂-C₃₀ fatty acid monoglyceride is understood to be the monoester of the trihydric alcohol glycerol with one equivalent of fatty acid. Either the middle hydroxy group of glycerol or the terminal hydroxy group of glycerol may be esterified with the fatty acid.

C₁₂-C₃₀ fatty acid monoglyceride is exemplified by its special suitability, in which a hydroxy group of glycerol is esterified with a fatty acid, whereby the fatty acids are selected from dodecanoic acid (lauric acid), tetradecanoic acid (myristic acid), hexadecanoic acid (palmitic acid), tetracosanoic acid (lignoceric acid), octadecanoic acid (stearic acid), eicosanoic acid (arachidic acid), docosanoic acid (behenic acid), petroselinic acid [(Z)-6-octadecenoic acid], palmitoleic acid [(9Z)-hexadec-9-enoic acid], oleic acid [(9Z)-octadec-9-enoic acid], elaidic acid [(9E)-octadec-9-enoic acid], erucic acid [(13Z)-docos-13-enoic acid], linoleic acid [(9Z,12Z)-octadeca-9,12-dienoic acid, linolenic acid [(9Z,12Z,15Z)-octadeca-9,12,15-trienoic acid, elaeostearic acid [(9Z,11E,13E)-octadeca-9,11,3-trienoic acid], arachidonic acid [(5Z,8Z,11Z,14Z)-cosa-5,8,11,14-tetraenoic acid] or nervonic acid [(15Z)-tetracos-15-enoic acid].

C₁₂-C₃₀ fatty acid diglyceride is the diester of the trihydric alcohol glycerol with two equivalents of fatty acid. Either the middle or one terminal hydroxy group of glycerol may be esterified with two equivalents of fatty acid, or both terminal hydroxy groups of glycerol are esterified with one fatty acid each. The glycerol may be esterified with two structurally identical fatty acids or with two different fatty acids.

Fatty acid diglycerides are particularly suitable for this purpose, in which at least one of the ester groups is formed from glycerol with a fatty acid selected from dodecanoic acid (lauric acid), Tetradecanoic acid (myristic acid), hexadecanoic acid (palmitic acid), tetracosanoic acid (lignoceric acid), octadecanoic acid (stearic acid), eicosanoic acid (arachidic acid), docosanoic acid (behenic acid), petroselinic acid [(Z)-6-octadecenoic acid], palmitoleic acid [(9Z)-hexadec-9-enoic acid], oleic acid [(9Z)-octadec-9-enoic acid], elaidic acid [(9E)-octadec-9-enoic acid], erucic acid [(13Z)-docos-13-enoic acid], linoleic acid [(9Z,12Z)-octadeca-9,12-diene acid, linolenic acid [(9Z,12Z,15Z)-octadeca-9,12,15-trienoic acid, elaeostearic acid [(9Z,11E,13E)-octadeca-9,11,3-trienoic acid], arachidonic acid [(5Z,8Z,11Z,14Z)-cosa-5,8,11,14-tetraenoic acid] and/or nervonic acid [(15Z)-tetracos-15-enoic acid].

Hydrocarbons are compounds with from about 8 to about 80 C atoms that exclusively include the atoms carbon and hydrogen. In this context, preference is given to aliphatic hydrocarbons such as mineral oils, liquid paraffin oils (e.g. paraffinium liquidum or paraffinum perliquidum), isoparaffin oils, semi-solid paraffin oils, paraffin waxes, hard paraffin (paraffinum solidum), Vaseline and polydecenes.

Liquid paraffin oils (paraffinum liquidum and paraffinium perliquidum) have proven to be particularly suitable in this context. Paraffinum liquidum, also known as white oil, is the preferred hydrocarbon. Paraffinum liquidum is a mixture of purified, saturated, aliphatic hydrocarbons, which mainly include hydrocarbon chains with a C chain distribution of from about 25 to about 35 C atoms.

Surfactants are surface-active substances that may form adsorption layers on surfaces and interfaces or aggregate in volume phases to form micelle colloids or lyotropic mesophases. A distinction is made between anionic surfactants including a hydrophobic residue and a negatively charged hydrophilic head group, amphoteric surfactants, which carry both a negative and a compensating positive charge, cationic surfactants, which in addition to a hydrophobic residue have a positively charged hydrophilic group, and non-ionic surfactants, which have no charges but strong dipole moments and are strongly hydrated in aqueous solution.

Within the context of a further explicitly particularly preferred embodiment, a product as contemplated herein is exemplified by the fact that it includes at least one anionic surfactant, at least one non-ionic surfactant, at least one amphoteric surfactant, at least one zwitterionic surfactant and/or at least one cationic surfactant.

Products preferred as contemplated herein additionally includes at least one anionic surfactant. Preferred anionic surfactants are fatty acids, alkyl sulfates, alkyl ether sulfates and ether carboxylic acids with from about 10 to about 20 C atoms in the alkyl group and up to about 16 glycol ether groups in the molecule. The anionic surfactants are used in quantities of from about 0.1 to about 45% by weight, preferably from about 1 to about 30% by weight and particularly preferably from about 1 to about 15% by weight, based on the total quantity of the product.

The product preferred as contemplated herein additionally includes at least one zwitterionic surfactant. Preferred zwitterionic surfactants are betaines, N-alkyl-N,N-dimethylammonium-glycinate, N-acyl-aminopropyl-N,N-dimethylammonium glycinate, and 2-alkyl-3-carboxymethyl-3-hydroxyethyl-imidazolines. A preferred zwitterionic surfactant is known under the INCI name cocamidopropyl betaine.

The product preferred as contemplated herein is exemplified by the fact that it additionally includes at least one amphoteric surfactant. Preferred amphoteric surfactants are N-alkylglycines, N-alkylpropionic acids, N-alkylaminobutyric acids, N-alkyliminodipropionic acids, N-hydroxyethyl-N-alkylamidopropylglycines, N-alkyltaurines, N-alkylsarcosines, 2-alkylaminopropionic acids and alkylaminoacetic acids. Particularly preferred amphoteric surfactants are N-coconut alkylaminopropionate, as coconut acylamino ethylamino propionate and C₁₂-C₁₈ acyl sarcosine.

The anionic, non-ionic, amphoteric, zwitterionic and/or cationic surfactants may be used in proportions of from about 0.1 to about 40% by weight, preferably from about 1 to about 30% by weight and very particularly preferably from about 1 to about 15% by weight, in each case based on the total amount of the product as contemplated herein.

Suitable solvents as contemplated herein may, for example, be selected from the group of ethanol, isopropanol, benzyl alcohol, phenoxyethanol, glycerol and 1,2-propanediol.

The product as contemplated herein may optionally also contain other active ingredients, auxiliaries substances and additives, such as non-ionic polymers such as vinylpyrrolidone/vinyl acrylate copolymers, polyvinylpyrrolidinone, vinylpyrrolidone/vinyl acetate copolymers, polyethylene glycols and polysiloxanes; additional silicones such as volatile or non-volatile, straight-chain, branched or cyclic, cross-linked or non-cross-linked polyalkylsiloxanes (such as dimethicones or cyclomethicones), polyarylsiloxanes and/or polyalkylarylsiloxanes, in particular polysiloxanes with organofunctional groups, such as substituted or unsubstituted amines (amodimethicones), carboxyl, alkoxy and/or hydroxyl groups (dimethicone copolyols), linear polysiloxane(A)-polyoxyalkylene(B) block copolymers, grafted silicone polymers; cationic polymers such as quaternized cellulose ethers, polysiloxanes with quaternary groups, dimethyldiallylammonium chloride polymers, acrylamide dimethyldiallylammonium chloride copolymers, dimethylamino ethylmethacrylate vinylate—vinylpyrrolidinone copolymers quaternized with diethyl sulfate, vinylpyrrolidinone imidazolinium methochloride copolymers and quaternized polyvinyl alcohol zwitterionic and amphoteric polymers; anionic polymers such as, for example, polyacrylic acids or cross-linked polyacrylic acids; structurants such as glucose, maleic acid and lactic acid, hair-conditioning compounds such as phospholipids, for example lecithin and cephalins; perfume oils, dimethyl isosorbide and cyclodextrins; fiber structure-improving active substances, in particular mono-, di- and oligo-saccharides such as glucose, galactose, fructose, fructose and lactose; dyes for coloring the composition; anti-dandruff active substances such as piroctone olamines, zinc omadine and climbazole; amino acids and oligopeptides; protein hydrolysates on animal and/or vegetable basis, such as in the form of their fatty acid synthesis products or optionally anionically or cationically modified derivatives; vegetable oils; light protection agents and UV blockers; active ingredients such as panthenol, panthenic acid, pantolactone, allantoin, pyrrolidinone carboxylic acids and their salts, and bisabolol; polyphenols, in particular hydroxy cinnamic acids, 6,7-dihydroxy coumarins, hydroxy benzoic acids, catechins, tannins, leucoanthocyanidins, anthocyanidins, flavanones, flavones and flavonols; ceramides or pseudo-ceramides; vitamins, provitamins and vitamin precursors; plant extracts; fats and waxes such as fatty alcohols, beeswax, montan wax and paraffins; swelling and penetrating substances such as glycerol, propylene glycol monoethyl ether, carbonates, hydrogen carbonates, guanidines, urea as well as primary, secondary and tertiary phosphates; opacifiers such as latex, styrene/PVP and styrene/acrylamide copolymers; pearlescent agents such as ethylene glycol mono- and distearate and PEG-3 distearate; pigments and propellants such as propane-butane mixtures, N₂O, dimethyl ether, CO₂ and air.

The selection of these other substances will be made by the expert according to the desired properties of the product. With regard to other optional components and the quantities of these components used, explicit reference is made to the relevant handbooks known to the expert. The additional active ingredients and auxiliary substances are preferably used in the agents as contemplated herein in quantities of from about 0.0001 to about 25% by weight each, in particular from about 0.0005 to about 15% by weight, each based on the total weight of the product as contemplated herein.

### Multi-Component Packaging Unit (Kit-of-Parts)

Shortly before use, the product as contemplated herein is mixed with an oxidizing agent preparation. In this way, the oxidative dye ready for use is obtained.

A second subject-matter of the present disclosure is therefore a multi-component packaging unit (kit-of-parts) for oxidative dyeing of keratinous fibers, comprising two separately packaged preparations (Z1) and (Z2), wherein

the preparation (Z1) is a product as disclosed in detail in the description of the first subject-matter of the present disclosure, and

the preparation (Z2) contains hydrogen peroxide in an aqueous cosmetic carrier.

The preparation (Z1), i.e. the product of the first present disclosure as contemplated herein, may alternatively be referred to as a color cream. The preparation (Z2) is the oxidizing agent preparation containing hydrogen peroxide in an aqueous cosmetic carrier.

In a preferred embodiment, hydrogen peroxide itself is used as an aqueous solution in the oxidizing agent preparation (K2). The concentration of a hydrogen peroxide solution in the dyeing preparation (K2) is determined on the one hand by the legal requirements and on the other hand by the desired effect; preferably from about 6 to about 12% by weight solution in water is used. Preparations (K2) preferred as contemplated herein include from about 5 to about 20% by weight, preferably from about 1 to about 12.5% by weight, particularly preferably from about 2.5 to about 10% by weight and in particular from about 3 to about 6% by weight of hydrogen peroxide, in each case based on the total weight of the oxidizing agent preparation (K2).

The dyeing preparation (K1) and/or the oxidizing agent preparation (K2) may contain further bleaching intensifiers, such as tetraacetylethylenediamine (TAED), to enhance the brightening effect, 1,5-diacetyl-2,4-dioxohexahydro-1,3,5-triazine (DADHT), tetraacetylglycoluril (TAGU), N-nonanoylsuccinimide (NOSI), n-nonanoyl- or isononanoyloxybenzenesulfonate (n- or i-NOBS), phthalic anhydride, triacetin, ethylene glycol diacetate and 2,5-diacetoxy-2,5-dihydrofuran as well as carbonate salts or hydrogen carbonate salts, in particular ammonium hydrogen carbonate, ammonium carbonate, sodium hydrogen carbonate, disodium carbonate, potassium hydrogen carbonate, dipotassium carbonate and calcium carbonate, and nitrogen-containing salts, heterocyclic bleaching intensifiers, such as 4-acetyl-1-methylpyridinium p-toluene sulfonate, 2-acetyl-1-methylpyridinium p-toluene sulfonate, and N-methyl-3,4-dihydro-isoquinolinium p-toluene sulfonate.

To further increase the brightening effect, the dyeing preparation (K1) and/or the oxidizing agent preparation (K2) may additionally contain at least one SiO₂ compound, such as silicic acid or silicates, especially water glasses. The SiO₂ compound may be contained in the dyeing preparation (K1) and/or in the oxidizing agent preparation (K2). As contemplated herein, it may be preferred to use the SiO₂ compounds in quantities of from about 0.05% by weight to about 15% by weight, particularly preferred in quantities of from about 0.15% by weight to about 10% by weight and very particularly preferred in quantities of from about 0.2% by weight to about 5% by weight, in each case based on the total weight of the dyeing preparation (K1) or on the total weight of the oxidizing agent preparation (K2). The quantities indicated in each case reflect the content of the SiO₂ compounds (without their water content) in the products.

The oxidative color changing agents (i.e. the dyeing preparation (K1) and/or the oxidizing agent preparation (K2)) may also contain additional active ingredients, auxiliary substances and additives to improve the dyeing or brightening performance and to adjust other desired properties of the product.

As further optional ingredients, which may additionally be contained in the oxidizing agent preparation (K2), the expert may, for example, select the additional active ingredients or additives which were also described in connection with the first subject-matter of the present disclosure.

### Method

The product as contemplated herein shows an extraordinary good suitability for dyeing keratinous fibers, whereby the dyed fibers showed very high color intensities and also very good fastness properties.

The fastness to washing of a color shade is understood to be the change in color of the hair strand dyed with this shade when subjected to several washes. This color change may be a shift of the color towards another shade as well as a fading of the coloration. Both color changes are equally undesirable for the user. Color shades with good washability do not or hardly change color even after repeated hair washes. The hair may be washed with the help of a shampoo, a conditioning shampoo or a conditioner.

The light fastness of a color shade is the change in color of the strand of hair dyed with this shade as a result of the influence of daylight (i.e. sunlight or UV or UV/Vis radiation). Irradiation with daylight usually causes the colored hair to fade. Nuances with good light fastness do not show any visible fading of the coloration even after several days of exposure to sunlight.

A third subject-matter of the present disclosure is therefore a method for dyeing keratinous fibers, in which a cosmetic product obtained by mixing a first preparation (Z1) with a second preparation (Z2) is applied to the keratinous fibers and rinsed out of the keratinous fibers after an exposure time of from about 5 to about 50 minutes, wherein

the first preparation (Z1) is a product as disclosed in detail in the description of the first subject-matter of the present disclosure, and

the second preparation (Z2) contains hydrogen peroxide in an aqueous cosmetic carrier.

With respect to other preferred embodiments of the kit and method as contemplated herein, the following applies mutatis mutandis to the product as contemplated herein.

### Examples

### 1. Production of the Coloring Agents

The following color creams were produced (unless otherwise indicated, all figures are in % by weight):

| Color creams | V1 | E1 |
|---|---|---|
| Isopropanol | 15.0 | 15.0 |
| Oleic acid, potassium salt | 6.0 | 6.0 |
| 1,2-propandiol | 6.0 | 6.0 |
| Lorol techn. (C₁₂-C₁₈ fatty alcohols) | 6.0 | 6.0 |
| Texapon NSO (sodium laureth sulfate, | 3.5 | 3.5 |
| approx. 27.5% by weight active | | |
| substance) | | |
| Dehydol LS 2 Deo N (BASF, fatty | 12.0 | 12.0 |
| alcohols C₁₂-C₁₄, ethoxylated (2 EO) | | |
| 1-hydroxyethane-1,1-diphosphonic acid | 0.12 | 0.12 |
| Vitamin C | 0.4 | 0.4 |
| Sodium sulfite | 0.4 | 0.4 |
| Ammonia | ad pH 10.0 | — |
| 2-(dimethylamino)-2-methyl-1-propanol | — | ad pH 10.0 |
| p-toluenediamine, sulfate | 0.66075 | 0.66075 |
| 1,5-dihydroxynaphthalene | 0.48051 | 0.48051 |
| Water (dist.) | ad 100 | ad 100 |
| Color creams | V2 | E2 |
| Isopropanol | 15.0 | 15.0 |
| Oleic acid, potassium salt | 6.0 | 6.0 |
| 1,2-propandiol | 6.0 | 6.0 |
| Lorol techn. (C₁₂-C₁₈ fatty alcohols) | 6.0 | 6.0 |
| Texapon NSO (sodium laureth sulfate, | 3.5 | 3.5 |
| approx. 27.5% by weight active | | |
| substance) | | |
| Dehydol LS 2 Deo N (BASF, fatty | 12.0 | 12.0 |
| alcohols C₁₂-C₁₄, ethoxylated (2 EO) | | |
| 1-hydroxyethane-1,1-diphosphonic acid | 0.12 | 0.12 |
| Vitamin C | 0.4 | 0.4 |
| Sodium sulfite | 0.4 | 0.4 |
| Ammonia | ad pH 10.0 | — |
| 2-(dimethylamino)-2-methyl-1-propanol | — | ad pH 10.0 |
| 2-(2-hydroxy-ethyl)-p-phenylenediamine, | 0.75081 | 0.75081 |
| sulfate | | |
| 1,5-dihydroxynaphthalene | 0.48051 | 0.48051 |
| Water (dist.) | ad 100 | ad 100 |
| Color creams | V3 | E3 |
| Isopropanol | 15.0 | 15.0 |
| Oleic acid, potassium salt | 6.0 | 6.0 |
| 1,2-Propandiol | 6.0 | 6.0 |
| Lorol techn. (C₁₂-C₁₈ fatty alcohols) | 6.0 | 6.0 |
| Texapon NSO (sodium laureth sulfate, | 3.5 | 3.5 |
| approx. 27.5% by weight active | | |
| substance) | | |
| Dehydol LS 2 Deo N (BASF, fatty | 12.0 | 12.0 |
| alcohols C₁₂-₁₄, ethoxylated (2 EO) | | |
| 1-hydroxyethane-1,1-diphosphonic acid | 0.12 | 0.12 |
| Vitamin C | 0.4 | 0.4 |
| Sodium sulfite | 0.4 | 0.4 |
| Ammonia | ad pH 10.0 | — |
| 2-(dimethylamino)-2-methyl-1-propanol | — | ad pH 10.0 |
| p-toluenediamine, sulfate | 0.66075 | 0.66075 |
| 2,6-dihydroxy-3,4-dimethylpyridine | 0.41748 | 0.41748 |
| Water (dist.) | ad 100 | ad 100 |

V = comparison
E = an embodiment as contemplated herein

The color creams V1, V2, V3, E1, E2 and E3 were each mixed in a 1:1 weight ratio with the following oxidizing agent preparation.

| Oxidizing agent preparation | OX |
|---|---|
| Dipicolinic acid | 0.10 |
| Sodium pyrophosphate | 0.03 |
| Turpinal SL (1-hydroxyethane-1,1-diphosphonic acid, | 1.50 |
| 58-61% by weight active substance) | |
| Texapon N28 (sodium laureth sulfate, at least 26.5% by | 2.00 |
| weight active substance) | |
| Dimethicone 10% | 0.067 |
| Aculyn 33 A (ACRYLATES COPOLYMER, ISP Global) | 12.0 |
| CAS No. 37325-11-4 | 12.0 |
| Hydrogen peroxide (50% aqueous solution) | 0.65 |
| Caustic soda (50% aqueous solution) | ad 100 |

### 2. Application

The ready-to-use oxidative dyes (V+OX and E+OX) produced in this way were applied to one strand of hair (Kerling, Euronaturhaar weiB and Kerling 9-0) and left to act for 30 minutes at room temperature. The strands were then rinsed with lukewarm tap water for one minute and dried in a cold air stream.

### 3. Measurement of the Color Intensities

After drying, the hair strands were left to rest for 12 hours and then measured colorimetrically (D65, 10 DEG).

From the L*a*b* values obtained, the color difference (ΔE value) between the undyed and the dyed strand was determined using the CIELAB formula. The ΔE values were used to evaluate the color intensity, whereby the higher the corresponding ΔE value, the higher the color intensity.

Measurement of color intensity on Kerling Euronaturhaar weiB (ENH), ΔE value (color difference uncolored-colored)

| | CIE L | CIE a | CIE b | ΔE value |
|---|---|---|---|---|
| ENH undyed | 72.53 | 3.06 | 25.77 | |
| V1 + OX | 42.79 | 4.47 | 10.85 | 33.31 |
| E1 + OX | 23.82 | 1.97 | −7.18 | 58.82 |
| V2 + OX | 41.84 | 4.67 | 9.33 | 34.85 |
| E2 + OX | 34.02 | 3.43 | −0.12 | 46.41 |
| V3 + OX | 50.17 | 2.52 | 15.29 | 24.70 |
| E3 + OX | 44.95 | 5.22 | 14.75 | 29.78 |

Measurement of color intensity on Kerling 9-0, ΔE value (color difference uncolored-colored)

| | CIE L | CIE a | CIE b | ΔE value |
|---|---|---|---|---|
| Kerling 9-0 undyed | 43.35 | 7.27 | 18.09 | |
| V1 + OX | 34.88 | 5.62 | 11.46 | 10.88 |
| E1 + OX | 24.63 | 2.27 | 1.14 | 25.74 |
| V2 + OX | 38.27 | 6.89 | 15.66 | 6.66 |
| E2 + OX | 27.34 | 4.41 | 3.55 | 21.82 |
| V3 + OX | 45.65 | 5.76 | 19.33 | 3.02 |
| E3 + OX | 37.63 | 5.72 | 15.28 | 6.56 |

The strands of hair treated with the oxidative dye (E+OX) as contemplated herein showed a higher ΔE value and thus higher color intensity compared to the comparison formulations (V+OX).

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth in the appended claims.

1. Product for the oxidative dyeing of keratinous fibers, included in a cosmetic carrier, wherein the product comprises:
(A) at least one developer selected from the group of p-toluenediamine, 2-(2-hydroxyethyl)-p-phenylenediamine, N,N-bis-(2-hydroxyethyl)-p-phenylenediamine, 2-methoxymethyl-p-phenylenediamine, p-aminophenol, 4-amino-3-methyl phenol, bis-(2-hydroxy-5-aminophenyl)-methane, 4,5-diamino-1-(2-hydroxyethyl)pyrazole or their physiologically compatible salts, and
(B) at least one coupler selected from the group of 1,5-dihydroxynaphthalene, 2,7-dihydroxynaphthalene, 1-naphthol, 1,7-dihydroxynaphthalene, 2,6-dihydroxy-3,4-dimethylpyridine, 2-amino-3-hydroxypyridine, 3-amino-2-methylamino-6-methoxypyridine, 3-aminophenol, 5-amino-2-methyl phenol, 3-amino-2-chloro-6-methyl phenol, 5-(2-hydroxy-ethyl)-amino-2-methyl phenol, 3-phenylenediamine, 2-(2,4-diaminophenoxy)ethanol, 1,3-bis(2,4-diaminophenoxy)propane, 1-methoxy-2-amino-4-(2-hydroxyethyl)amino)benzene, 2,6-bis(2′-hydroxyethylamino)-1-methylbenzene, resorcinol, 2-methylresorcinol, 4-chlororesorcinol or their physiologically compatible salts, and
(C) 2-(dimethylamino)-2-methyl-1-propanol.

2. Product according to claim 1, comprising:
(A) at least one developer selected from the group of p-toluenediamine, 2-(2-hydroxyethyl)-p-phenylenediamine or their physiologically compatible salts.

3. Product according to claim 1, comprising:
(A) at least one developer selected from the group of p-aminophenol, 4-amino-3-methyl phenol or their physiologically compatible salts.

4. Product according to claim 1, comprising—based on the total weight of the product—one or several developers (A) in a total amount or from about 0.01 to about 5.0% by weight.

5. Product according to claim 1, comprising:
(B) at least one coupler selected from the group of 1,5-dihydroxynaphthalene, 2,7-dihydroxynaphthalene, 1-naphthol or 1,7-dihydroxynaphthalene.

6. Product according to claim 1, comprising:
(B) at least one coupler selected from the group of 2,6-dihydroxy-3,4-dimethylpyridine, 2-amino-3-hydroxypyridine, 3-amino-2-methylamino-6-methoxypyridine or their physiologically compatible salts.

7. Product according to claim 1, comprising:
(B) at least one coupler selected from the group of 3-aminophenol, 5-amino-2-methyl phenol, 3-amino-2-chloro-6-methyl phenol, 5-(2-hydroxyethyl)-amino-2-methyl phenol or their physiologically compatible salts.

8. Product according to claim 1, comprising:
(B) at least one coupler selected from the group of 3-phenylenediamine, 2-(2,4-diamino-phenoxy)ethanol, 1,3-bis(2,4-di¬aminophenoxy)propane, 1-methoxy-2-amino-4-(2-hydroxyethylamino)benzene, 2,6-bis(2′-hydroxyethylamino)-1-methylbenzene or their physiologically compatible salts.

9. Product according to claim 1, comprising:
(B) at least one coupler selected from the group of resorcinol, 2-methylresorcinol or 4-chlororesorcinol salts.

10. Product according to claim 1, comprising—based on the total weight of the product—one or several couplers (B) in a total amount from about 0.01 to about 5.0% by weight.

11. Product according to claim 1, comprising—based on the total weight of the product—from about 0.5 to about 25.0% by weight of 2-(dimethylamino)-2-methyl-1-propanol (C).

12. Product according to claim 1, comprising—based on the total weight of the product—less than about 1.75% by weight of ammonia.

13. Product according to claim 1, comprising water and having a pH value of 8.0 to 12.0.

14. Product according to claim 1, further comprising at least one further ingredient selected from the group of fatty constituents, surfactants and/or solvents.

15. Multi-component packaging unit (kit-of-parts) for oxidative dyeing of keratinous fibers, comprising two separately packaged preparations (Z1) and (Z2), wherein
preparation (Z1) is a product according to claim 1, and
preparation (Z2) comprises hydrogen peroxide in an aqueous cosmetic carrier.

16. Method for coloring keratinous fibers, in which a cosmetic product obtained by mixing a first preparation (Z1) with a second preparation (Z2) is applied to the keratinous fibers and, after a contact time of from about 5 to about 50 minutes, is rinsed out of the keratinous fibers, wherein
the first preparation (Z1) is a product according to claim 1, and
the second preparation (Z2) comprises hydrogen peroxide in an aqueous cosmetic carrier.

17. Product according to claim 1, comprising:
(A) at least one developer selected from the group of p-toluenediamine, 2-(2-hydroxyethyl)-p-phenylenediamine and their physiologically compatible salts; and
(B) at least one coupler selected from the group of 1,5-dihydroxynaphthalene or 2,6-dihydroxy-3,4-dimethylpyridine.

18. Product according to claim 1, comprising—based on the total weight of the product:
one or several developers (A) in a total amount from about 0.5 to about 0.8% by weight; and
one or several couplers (B) in a total amount from about 0.4 to about 0.8% by weight.

19. Product according to claim 1, comprising—based on the total weight of the product—from about 8.0 to about 14.0% by weight of 2-(dimethylamino)-2-methyl-1-propanol (C).

20. Product according to claim 1, comprising:
(A) at least one developer selected from the group of p-toluenediamine, 2-(2-hydroxyethyl)-p-phenylenediamine and their physiologically compatible salts in a total amount from about 0.5 to about 0.8% by weight; and
(B) at least one coupler selected from the group of 1,5-dihydroxynaphthalene or 2,6-dihydroxy-3,4-dimethylpyridine in a total amount from about 0.4 to about 0.8% by weight;
wherein the 2-(dimethylamino)-2-methyl-1-propanol (C) is present—based on the total weight of the product—in an amount of from about 8.0 to about 14.0% by weight.
